(19) United States
(12) Patent Application Publication
(10) Pub. No.: US 2023/0355517 A1
(43) Pub. Date: Nov. 9, 2023

(54) USE OF ACTIVE SUBSTANCES WITH ANTIVIRAL, ANTI MALARIAL, AND/OR MUCOLYTIC PROPERTIES IN THE TREATMENT OF VIRAL LUNG DISEASES INCLUDING COVID-19 BY SOFT MIST INHALER OR VIBRATION MESH TECHNOLOGY NEBULIZER THROUGH INHALATION ROUTE

(71) Applicant: ATANBUL UNIVERSITESI REKTOLUGU, Fatih/Istanbul (TR)

(72) Inventors: Ayca Yildiz PEKOZ, Fatih/Istanbul (TR); Ahment Ogul ARAMAN, Fatih/Istanbul (TR); Ozlem Akbal DAGISTAN, Fatih/Istanbul (TR)

(21) Appl. No.: 18/245,053

(22) PCT Filed: Jul. 14, 2021
(86) PCT No.: PCT/TR2021/050733

§ 371 Date: Mar. 13, 2023

(30) Foreign Application Priority Data
Sep. 14, 2020 (TR) 2020/14543
Dec. 10, 2020 (TR) 2020/20261
Jan. 5, 2021 (TR) 2021/00055
Jan. 13, 2021 (TR) 2021-11493

(51) Int. Cl. A61K 9/00; A61K 31/4965; A61K 31/047; A61K 31/4706; A61K 31/4045; A61K 31/706; A61M 11/00

(52) U.S. Cl. CPC A61K 9/0078; A61K 31/4965; A61K 31/047; A61K 31/4706; A61K 31/4045; A61K 31/706; A61M 11/003

(57) ABSTRACT

The present invention relates to the administration of active substances with antiviral, antimalarial and/or mucolytic properties, or pharmaceutically acceptable derivatives thereof for the treatment of viral lung diseases, especially COVID-19 by means of soft mist inhaler or vibrating mesh technology (VMT) nebulizer through inhalation. The present invention particularly relates to the administration of favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms obtained by water-solubility increasing methods in the treatment of viral lung diseases, especially COVID-19 by means of soft mist inhaler or vibrating mesh technology (VMT) nebulizer through inhalation. Active substances with antiviral, antimalarial, and/or mucolytic properties reach the lungs efficiently and quickly, and local pulmonary administration is performed such that it provides an effective treatment. Since the drug is targeted directly to the lungs without getting into systemic circulation via local (direct) administration, its concentration is higher at the application region, thereby reducing the side effects and costs per application of the drug, and increasing its efficacy.

### TECHNICAL FIELD OF THE INVENTION

The present invention relates to the administration of active substances with antiviral, antimalarial and/or mucolytic properties, or pharmaceutically acceptable derivatives thereof for the treatment of viral lung diseases, especially COVID-19 by means of soft mist inhaler or vibrating mesh technology (VMT) nebulizer through inhalation.

The present invention particularly relates to the administration of favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms obtained by water-solubility increasing methods in the treatment of especially COVID-19, viral lung diseases, acute and/or chronic lung diseases by means of soft mist inhaler or vibrating mesh technology (VMT) nebulizer through inhalation.

### STATE OF THE ART

Coronaviruses (CoV) are a large family of viruses that cause diseases ranging from the common cold to more serious diseases such as Middle East Respiratory Syndrome (MERS-CoV) and Severe Acute Respiratory Syndrome (SARS-CoV). Coronaviruses are single-stranded, positive-polarity, enveloped RNA viruses. They have rod-like extensions (protrusions) on their surfaces. The Latin equivalent of the crown-like structure formed by these protrusions is “corona”, and based on this, these viruses were named Coronavirus (coronavirus, crowned virus). Coronaviruses are classified into four main genera alpha-, beta-, gamma- and delta coronaviruses. They can be detected in humans, domestic and wild animals (bat, camel, pig, cat, dog, rodent and poultry, etc.). Human coronaviruses were first identified in the 1960s. Today, there are seven coronaviruses known to have infection factors in humans. 229E (Alpha coronavirus), NL63 (Alpha coronavirus), OC43 (Beta coronavirus), and HKU1 (Beta coronavirus) are the coronaviruses that are the most common infectious factors in humans and affect the upper and lower respiratory tract. Three other human coronaviruses have been identified recently, and they are; SARS-CoV, MERS-CoV, and lastly SARS-CoV-2. SARS-CoV virus has been identified in 2002 in China. It causes Severe Acute Respiratory Syndrome (SARS). The epidemic caused the death of 774 people worldwide. MERS-CoV emerged in Saudi Arabia in 2012 and was named Middle East Respiratory Syndrome virus (MERS), it spread to 24 countries, and caused more than 1000 cases and around 400 deaths. SARS-CoV-2, on the other hand, is an infectious and extremely pathogenic coronavirus that started in Wuhan, Hubei province of China in the last days of December 2019, and caused pneumonia in humans, an epidemic of severe respiratory tract infection, and subsequently spread first across the country, and then all over the world. The epidemic was initially detected in people who are in the seafood and animal market in this region; however, it later spread from person to person and spread to other cities in Hubei province, especially Wuhan, to other provinces of the People's Republic of China, and to other countries in the world due to the interaction between people and travels. Since December 2019, when the virus first appeared, the world population has almost quarantined globally, and it caused a global economic slowdown. Since there is no full treatment, how long this quarantine will last and its negative effect on people and economies is unpredictable; it caused the death of more than 3.8 million people until July 2021, and it is stated that this number will exceed millions until herd immunity is achieved.

This new viral respiratory disease caused by the SARS-CoV-2 virus and the most common symptoms thereof, which manifest themselves as high fever, cough, and respiratory distress (dyspnea, difficulty in breathing), has been defined as COVID-19 by the World Health Organization. The SARS-CoV-2 virus directly targets the lungs, and lung destruction begins in a short time as 5 days. Patients generally die due to respiratory failure. At present, a drug capable of completely treating COVID-19 clinically is not available. The drugs currently used are antivirals, cytokine inhibitors, and antibody administration methods, which are used in the palliative treatments of previous viruses. COVID-19 is transmitted by means of coughing/sneezing of sick individuals and inhaling droplets spread in the environment. People might get infected with the virus in case they touch their face, eyes, nose or mouth without washing/disinfecting them after touching surfaces contaminated with respiratory particles of sick people. For this reason, touching the eyes, nose or mouth with dirty hands during this epidemic carries a great risk. The incubation period of the SARS-CoV-2 coronavirus is between 2 days and 14 days, and milder complaints (such as fever, sore throat, weakness) are observed in the first few days of the disease, and after then, symptoms, which manifest themselves as cough and difficulty in breathing (dyspnea) are observed, and conditions of patients usually become severe enough to apply to the hospital after 7 days. In consideration of the data obtained, the virus contains a higher risk of causing severe disease for people of advanced age (65 years and older) and accompanying disease (asthma, diabetes, heart disease, etc.). Some of the people infected with SARS-CoV-2 coronavirus survive the disease mildly and do not show symptomatic indications, however, since said individuals are the carriers, they carry the disease to the people they get in contact with. Carrier patients generally are children and young individuals. Although the current data indicate that the mortality rate of the disease is around 2%, but said information may differ depending on the changes that may occur in the genetic structure of the virus. In severe cases, pneumonia, severe acute respiratory tract infection, severe respiratory failure, kidney failure and even death may occur.

It is known that viral infection affects the respiratory system and cardiac system with the pathogenesis of the SARS-CoV-2 virus starting in the body. Data obtained from the cohort and the autopsies of deceased patients indicate that people infected with the SARS-CoV-2 virus develop a coagulopathic profile. A multicenter retrospective cohort study conducted in the People's Republic of China has included 191 adult patients who were proven to have COVID-19 through laboratory data. Coagulopathy was observed in 50% of patients who died. The rate of coagulopathy with sepsis complications was recorded as 70% in patients who died. In addition, coagulative abnormalities was observed in patients infected with COVID-19, but it has also been stated that these are not the typical disseminated intravascular coagulation (DIC) observed in sepsis. Furthermore, lung microthrombi formation was also confirmed in patients who were subjected to autopsy.

In addition to thrombus formation in patients infected with the SARS-CoV-2 virus, it is assumed that the procoagulant and anticoagulant state that is observed during infection triggers the balance disorder between immune and non-immune cells, and also triggers a thrombus formation. Endothelium plays a critical role in maintaining body homeostasis and it is known that viral infections will disrupt the integrity of the endothelium, and it causes a possible risk of hematopathology. Additionally, it is thought that von Willebrand factor elimination, T-like receptor activation, and tissue factor pathway activation that is induced as a result of viral infection play a role in the coagulant cascade together, and this effect causes cross-linked fibrin coagulation. Each physiological response for excessive activation of the coagulant cascade required for the destruction of these clots is responsible for the procoagulant D-dimer factor. Following antigen recognition, platelets are activated in addition to D-dimer, thereby allowing white blood cells to coordinate for the purpose of removing pathogens and forming coagulation. As a result, immune cells, platelets and endothelial cells play a role in the formation of the coagulopathic profile in viral infection. In addition to this clinical picture, it should be taking into consideration that the picture of venous thromboembolism will also constitute an additional reason in favor of coagulation since COVID-19 patients are on bed rest for a long time.

Pathogens such as viruses reach and settle the lungs through inhalation route, and cause severe infections in this region. Administration alternatives that are formulated in conventional dosage form and that have systemic effects are generally used in the treatment of these microbial and viral-based diseases that demonstrate high retention in the lung and that cause severe lung infections. The main disadvantages of the conventional dosage forms (tablets, parenteral drugs etc.) that are commercially available for use against the relevant symptoms in the treatment of acute diseases (COVID-19, pneumonia, etc.) or chronic diseases (COPD, asthma, etc.) in the lungs can be summarized as follows:
1) It has been proven that the accumulation of drugs administered in both tablet dosage form and parenteral form in the lung bronchoalveolar lavage fluid and lung tissue is low with respect to the lack of accumulation of the active substance in the lung tissue.
2) There is a possibility that the active substances used may have toxic effects on the whole body due to the systemic administration.
3) Since it is not possible for intubated patients to swallow drugs in tablet form and since it may be difficult for pediatric patients to swallow drugs in tablet form due to size of tablets, the common practice that is based on medical treatment guidelines in hospitals is administering drugs in tablet form by crushing them. However, although it is practical, said administration method causes antivirals to become unstable and their bioavailability to decrease by up to 50% due to especially crystal structures thereof.
4) Compliance with conventional dosage forms in pediatric patients is low due to the reasons such as bad taste of active substances and difficulty in swallowing. 5) Correct dose adjustment cannot be performed by dividing or crushing for the administration due to the fact that tablet forms are mostly film-coated.

The choice of a drug that is used in the treatment of lung diseases (as in any organ or tissue) is primarily for the local treatment of said organ or tissue. Local treatment ensures the drugs to be used are effective only in the determined organ or tissue, and other parts of the body are not exposed to the drug systemically. The administration results in more effective and the side effects thereof are reduced by means of the local administration of the drug, although the active substances are applied in lower amounts. Therefore, clinicians and researchers have turned to options of local application as an alternative to conventional dosage forms in the applications of lung diseases due to the disadvantages mentioned above.

COVID-19 pandemic necessitates dosage forms that may be formulated very quickly and technologies thereof. Inhalation devices used in the treatment of lung diseases are metered-dose inhaler (MDI), dry powder inhaler (DPI), nebulizers (Jet, ultrasonic, new type nebulizer (e.g. VMT and electronic)), and soft mist inhalers). The use of MDI and DPI's are not very advantageous, especially for patients with severe respiratory distress, and involve many drawbacks (difficulty of use, inability to control their activity, risk of contamination). The ideal drug accumulation in lung is difficult to achieve due to the high aerosol velocities of 2 m/s-8 m/s of MDIs containing hydrofluoroalkane. In addition, there is a practical difficulty for the patient to inhale the drug in a controlled manner simultaneously with device activation and at an appropriate slowness. It is difficult to provide that for COVID-19 patients and not practical. In DPIs, on the other hand, the fine particle dose is dependent on the respiration stream of air and absolute lung capacity, which is a parameter that varies widely according to patients. The use of DPIs in COVID-19 disease causes an increase in lung tenderness and collapse since said devices contain active substances and excipients in powder form. For the production of both of these types of devices, formulation of active substances considered in inhalation treatment for COVID-19 and starting to use them in patients require quite time-consuming production steps. Thus, it is more convenient to administer the formulation to be prepared through inhalation in the treatment of COVID-19 by means of nebulizers or soft mist inhalers, since it is a process that can be completed more quickly. Standard nebulizers are not safe for the treatment of COVID-19 due to tidal breathing, wide distribution of droplets, and also the reason of distribution risk of patient saliva by the nebulizer. Droplets released during exhalation spread the virus around. At this point, device selection becomes prominent. Standard nebulizers are not safe for COVID-19 patients due to common tidal breathing problems, wide distribution of droplets, distribution of patient saliva by the nebulizer, and posing a risk of infection for health care personnel. In practical terms, jet, ultrasonic, or electronic nebulizers cause distribution of the virus and pose a risk of infection, and they should not be preferred with regard to the wellness of health care personnel due to the fact that they cause physician and nurse deaths as observed in Italy and USA. Droplets scattered in breathing carry viruses and it is very important to minimize this risk during the treatment process. Therefore, choosing the right administration route and the right nebulizer is extremely important in the treatment of viral lung diseases including COVID-19 disease.

Soft mist inhaler (so named to describe aerosol production mechanisms and aerosol cloud properties) is a non-pressure metered dose inhaler that uses microfluidic technology and features a measuring function that enables to delivery of different doses (19-20). In DPIs, the fine particle dose produced is highly dependent on the inspiratory stream of air and absolute lung capacity, which varies widely according to patients (19). On the other hand, soft mist inhalers provide many advantages in terms of lung accumulation and ease of use. Soft mist inhalers are active systems that do not require propellant, in other words, the energy required for aerosol production is supplied from the inhaler and is therefore independent of the inspiratory capacity of the patient (20). Soft mist inhalers provide many more advantages in terms of drug accumulation in the lungs and ease of use. The soft mist inhaler works with an active mechanism that does not require propellant; the energy required for aerosol production is provided from the inhaler itself. Thus, the soft mist inhaler is independent of the patient's respiratory capacity. The solution must be converted into droplets in order to produce a respirable aerosol with an appropriate size from a drug solution by means of this system. As an operation principle, the soft mist inhaler creates a mechanical force by compressing the spring, and the driving force causes the piston is compressed, thereby triggering the drug solution to be passed through a series of small pores in order to form an aerosol. In the studies, in vitro physical methods such as droplet size, aerosol velocity during the droplet formation, and also imaging techniques that provide evaluation in vivo environment were utilized to support in vitro data in order to determine the deposition of the aerosol produced by means of soft mist inhalers in the peripheral airways. The size range of the aerosol droplets released from the device is in the range of 2-6 micrometers, and said aerosol droplets target the lungs. Another advantage of the soft mist inhaler is that dosing is performed by means of a syringe. The present parenteral form of the drug/active substances may be administered by integrating it into the soft mist inhaler without requiring an additional formulation step by means of said syringe system.

Dose-to-dose reproducibility of soft mist inhalers that enables delivering a drug in a solution form with a certain volume from a depot delivery system or a single use dosage form is more consistent than MDIs that release small amounts of suspension and DPIs that are carried in powder. In soft mist inhalers, the drug is in dissolved form in solution; therefore, it is affected less by moisture ingress compared to dry powders, thus soft mist inhalers are suitable for use in areas with humid environmental conditions. The relatively low velocity and long spray time of the soft mist inhaler facilitate the inhalation of the aerosol in a reproducible manner. However, there is often a requirement that the drug is soluble and stable in the solution for the soft mist inhalers unless certain formulation technologies are not applied.

Spiriva Respimat® (tiotropium) and Striverdi Respimat® (olodaterol) are the commercially available products as examples of soft mist inhaler formulations. In addition, in the state of the art, patent application numbered CN101773491A discloses an inhalation solution that is developed for use in the treatment influenza and that enables the administration of zanamivir, which is an active substance with antiviral activity, through inhalation route.

In the prior art, patent document numbered U.S. Ser. No. 10/328,128B2 discloses a preparation process of microparticle formulations containing Favipiravir and Umifenovir (Arbidol®) for the purpose of specifically treating enterovirus D68 through inhalation route. Said patent only comprises formulations prepared for the purpose of treating enterovirus D68. Patent application numbered CN111249229A includes the use of cyclodextrin complexes, but describes only the injection use of favipiravir cyclodextrin complex. In the state of the art, patent document numbered RU2593570C1 discloses the coated tablet form of the umifenovir active substance with antiviral and immunostimulatory activity. On the other hand, the patent application numbered RU2014141023A discloses the process of preparing the same active substance with the method of non-solvent deposition in nanocapsule form.

The US patent application numbered US2008138397A1 discloses formulations based on taste-masking of hydroxychloroquine substance during its administration in the form of liposome by the pulmonary/inhalation route either singularly and/or in combination in order to minimize the tendency to stimulate the cough reflex, and/or to minimize its retention in the oropharygeal area.

The patent application numbered CN111205327A, as another document in the state of the art discloses a synthesis method for the production of remdesivir active substance with antiviral effect. The different processes for preparing the same active substance with different synthesis methods are described in the patent applications numbered CN111116656A, CN111187269A, CN111233870A, and CN111233869A. Another patent document numbered CN111297838A in the prior art discloses inhalation spray drugs for administering antiviral drugs specifically with an atomizing spray device through inhalation route. Patent application numbered CN111320650A, on the other hand, relates to the production of remdesivir salt and its use in the treatment of coronavirus.

Today, drug applications with nebulizers are widely used in the treatment of respiratory diseases. Nebulization (Inhalation) treatment is a process applied in order to deliver drugs in liquid form directly to the respiratory tract and lungs by aerosolizing them with devices called nebulizers. There are several advantages of administering drugs with a nebulizer, such as not applying drugs with an invasive procedure, applying them directly to the lungs, its immediate onset of action, having less side effects, and not requiring hand-mouth coordination. Nebulizers are devices that convert the drugs produced for these devices into vapor form with sound waves, compressed air, or vibrations they create using electrical energy and that enable the drugs to be administered through inhalation route. There are special drug forms prepared in order to be used only in nebulizers, and these drugs are so-called nebul drugs. Three types of nebulizers are used in the nebulization of liquid drugs: ultrasonic, jet and mesh. Historically, jet nebulizers have been the standard delivery system for aerosol drugs. They are relatively inefficient and require an external air source to operate. Jet nebulizers are devices with compressors, they have a motor that produces compressed air, thereby convert the drug into vapor. Jet nebulizers with a corrugated tube are conventional constant-output nebulizers that continuously produce aerosols during inspiration. These nebulizers have several disadvantages. Limited portability, compressed air/gas sources for operation, and variability between them are some of the disadvantages thereof. The second type of nebulizer is the ultrasonic nebulizer. High frequency sound waves are created by means of the vibration of the piezoelectric crystal in this device. The drug or water is disintegrated into particles and vapor is released from the device by means of these sounds that the human ear cannot detect. Although the nebulizer with compressor is loud, the ultrasonic nebulizer operates in complete silence.

On the other hand, vibrating mesh technology was developed as an alternative to jet nebulizers. It is known that vibrating mesh technology nebulizers are more efficient than jet nebulizers and they do not require additional gas in the ventilator circuit. On the other hand, vibrating mesh nebulizers may be more sensitive to the contamination risk and device orientation and have precision electronic controls when compared to jet nebulizers. Vibrating mash technology (VMT) nebulizers provide many advantages with their consistent and improved aerosol production efficiency, fine particle fraction that can reach the peripheral lung, and nebulization capability in low residual volume and low drug volumes. VMT nebulizers are active systems that do not require propellant and that use micro-pump technology; and the energy required for aerosol production is provided from the inhaler in the physical mechanism. Therefore, drug delivery to the target region in the lungs is independent of the respiratory capacity of the patient. VMT nebulizers feature short processing times and silent operation. The pore size of VMT nebulizers may be optimized by adjusting the aerosol chamber and output rate for different drugs. VMT nebulizer, as a working principle, is based on the fact that thousands of holes on a membrane vibrate at the same time for hundreds of thousands of times per second, and the liquid that passes through these holes creates aerosol droplets with suitable size for targeting the drug to the lungs. The system control sensors detect if there is any liquid contact with the atomizing membrane, and allow the liquid to pass through thousands of holes created via precision laser by means of the vibrations in the resonant bending mode, thereby creating fine droplets having a narrower size distribution than the present systems. Membran can be designed so as to yield droplets with a certain size that are suitable for the physical properties of the solution by means of changing the pore size of said membrane. The VMT nebulizer ensures that the dosing is carried out in a much better way since there is no aerosol escape unlike conventional nebulizers (jet or ultrasonic) by means of its system that fits into the mouth and that is developed for maskless use. In addition, the room contamination problem observed in the use of the classical type nebulizer in the treatment of COVID-19 is no longer a problem since the VMT nebulizer works in a closed system by means of its mouthpiece. In VMT nebulizer, the drug is in dissolved form in solution, therefore, it is affected less by moisture ingress compared to dry powders, thus VMT nebulizers are suitable for use in humid environments. Another advantage of VMT nebulizers is that they facilitate the inhalation of aerosol in a reproducible manner by means of the long spraying time with the low velocity thereof. The drug to be applied in the vibrating mesh nebulizer is positioned on the concave side of the mesh and the mesh is vibrated at high frequency by using a piezoelectric actuator. This allows the drug to transform into a cloud consisting of small droplets that can be delivered from the bottom (convex) side of the mesh. In addition, the droplet size can be adjusted by means of said technology as mentioned above. In particular, geometrical changes can be performed to the mesh structure in order to provide a desired certain droplet size. The droplets may move away from the device under the force of gravity at low velocity due to the absence of atomization gas. In addition, the number of holes in the mesh and their placement on the mesh may also be customized.

The patent application numbered US2014020680A1 in the state of the art discloses a nebulizer device that allows for producing an aerosol cloud containing a therapeutic agent therein, and that operates with a vibrating mesh system and a drug administration method thereof. Said patent application comprises delivering amikacin and vancomycin antibacterials as a therapeutic agent.

Patent application numbered WO2017202885A1 relates to the administration of oseltamivir carboxylate in the treatment of viral infections through pulmonary route. On the other hand, the patent document numbered U.S. Pat. No. 6,572,858B1 describes local administration of the hydroxychloroquine, which is an anti-malarial active agent, for the treatment of inflammatory lung diseases such as chronic lung disease, asthma, and sarcoidosis. Here, eye drops, suppository, nasal spray, oral paste, and inhalation route are mentioned as a route of administration, and only the use of antimalarial drugs in the treatment of inflammatory diseases is included in the scope.

In the state of the art, patent application numbered WO2017085692A1 describes the administration of ribavirin active ingredient by inhalation and content of the formulation of the active ingredient to be administered in this way. Said document mentions dry powder inhaler as an inhale form.

The limitations and inadequacies of the available solutions in the current technique necessitated making an improvement for the effective treatment of viral lung diseases, especially COVID-19.

### BRIEF DESCRIPTION AND OBJECTS OF THE INVENTION

The present invention discloses the administration of favipiravir, mannitol, hydroxychloroquine, umifenovir, molnupiravir, pimodivir, and/or remdesivir, and/or pharmaceutically acceptable derivatives thereof, with antiviral, antimalarial, and/or mucolytic properties, especially their water-soluble salt forms, and/or their water-soluble cyclodextrin complexes, and/or their water-soluble forms obtained by water-solubility increasing methods by means of soft mist inhaler or vibrating mesh technology (VMT) nebulizer through inhalation route, compositions containing said active substance, and effective dosage forms and doses thereof. In the present invention, said active substances are administered locally and directly to the lung through pulmonary route. The pulmonary route is a suitable route for administering active substances with weaker absorption features than the oral route and with peptide-protein structures that are broken down in the stomach, or active substances that are rapidly metabolized. Pharmaceutical composition of the present invention, in addition to containing favipiravir, mannitol, hydroxychloroquine, umifenovir, molnupiravir, pimodivir, and/or remdesivir, and/or pharmaceutical derivatives thereof, may also contain different active substance(s), and/or excipient(s). In addition to antiviral agents, the involvement of mucolytic dosage form in the formula is a factor that improves the overall formula structure. The preparation of the hypertonic solution of the aforementioned antivirals is for the inhaled product developed to have both antiviral and mucolytic effects due to the mucolytic effect of the hypertonic solution. However, antivirals can also be used solely in said pharmaceutical composition.

The most important object of the present invention is to provide effective treatment of viral lung diseases, especially COVID-19. The present invention allows the active substance is administered locally (directly) to the lung in the treatment of viral lung diseases such that it has many advantages compared to the other administration routes (oral, parenteral, etc.), thereby, providing more effective treatment.

Another object of the present invention is to ensure that the drugs/active substances used in the treatment of viral lung diseases, especially COVID-19 are effective with higher efficacy and minimize the side effects thereof. In the present invention; the drug efficacy increases, and side effects of the drug, which may occur systemically are reduced by means of its local administration compared to the oral and parenteral routes.

Yet another object of the present invention is to provide effective treatment of viral lung diseases, especially COVID-19 by means of an application with high bioavailability. In the present invention, the administration of favipiravir, mannitol, hydroxychloroquine, umifenovir, molnupiravir, pimodivir, and/or remdesivir, and/or their pharmaceutical derivatives through pulmonary route increases the bioavailability since the effect of liver first pass is eliminated. This is because the pulmonary route is an optimal route of administration for drugs that are poorly absorbed or quickly metabolized through the oral route. Thus, the effect of liver first pass is prevented by means of the administration of drugs through pulmonary route. In addition, since the pass of macromolecular structures through the lungs is quite well, the effectiveness of the treatment is higher than the current administration methods.

Yet another object of the present invention is to treat the damage caused by the COVID-19 disease to the lungs. The present invention provides the treatment of viral lung diseases, especially COVID-19 disease caused by the SARS-CoV-2 virus by means of the administration of favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, or pharmaceutically acceptable derivatives thereof through pulmonary route.

Another object of the present invention is to minimize the infection risk for health care personnel and uninfected people in the environment during the treatment of especially COVID-19, and viral lung diseases. The risk of infection to the environment is reduced by means of the inhalation applications (soft mist inhaler or VMT nebulizer) of the present invention. The present invention enables the application such that the contamination of the room air is prevented by means of the closed system operation, and minimizes the environmental contamination caused by the patient's saliva.

In the present invention, accumulation (condensation of drug/active substance-containing solutions) in the environment and in the upper respiratory tract is minimized, and thus, an aerosol with a low velocity that optimizes drug accumulation is produced by means of the administration of favipiravir, mannitol, hydroxychloroquine, umifenovir, molnupiravir, pimodivir, and/or remdesivir, and/or pharmaceutical derivatives thereof via vibrating mesh nebulizers. Vibrating mesh technology nebulizers do not affect the stability of the drug/active substance since they do not generate heat.

In the present invention, drug localization in the lungs is much higher (20% and above) compared to other devices by means of the administration of favipiravir, mannitol, hydroxychloroquine, umifenovir, molnupiravir, pimodivir, and/or remdesivir, and/or pharmaceutical derivatives thereof via soft mist inhaler. The reason for this is that the droplet size range in a soft mist inhaler is so localized in the lungs that it is incomparable with a metered dose inhaler (MDI), dry powder inhaler (DPI), jet, or ultrasonic nebulizer. The mechanism of soft mist inhaler device is that it operates with the dosage form in the form of a solution, and the solution is fed into the device by means of a syringe. The syringe ensures that the therapeutic agents (active substances of the present invention) are administered in the patient-specific solution dosage form and with the appropriate sensitivity and frequency in terms of dose, based on the needs of the patient. In the soft mist inhaler, the user fits the device into his/her mouth via the mouth piece and inhales through mouth and subsequently, exhales through nose, thereby minimizing the risk of exhalation through the mouth. Environmental contamination of saliva is prevented by means of creating a closed system. The soft mist inhaler used in the present invention has an application apparatus attached to the intubation tube that is developed for intubated patients, and this attachment makes the inhaler superior compared to present inhalers. Said appliance provides ease of application for intubated patients and/or patient groups who cannot benefit from oral dosage forms that are difficult to swallow such as tablets and capsules, and the application according to the present invention offers high patient compliance. The present invention ensures that said active substances are used in solution dosage form specific to patient and with appropriate sensitivity and frequency in terms of dose. In other words, there are advantages such as increasing localization of lung, providing treatment with drugs at lower doses, eliminating the systemic side effects of locally used antivirals, providing inhaled treatment to patients who cannot take antiviral drugs orally, avoiding contamination risk, especially in pandemic by the closed system drug administration, and creating a synergistic effect by means of the synergistic effects thereof that allows both the formulation of the present invention is in the solution form as a dosage form and it is applied via VMT and soft mist.

Liquid dosage forms become prominent as the easiest and fastest formulation type among other dosage forms. Moreover, the production stages and requirements of liquid dosage forms may be completed in a shorter time than other dosage forms. The soft mist inhaler used in an application of the present invention works with a liquid dosage form. Thus, the formulation may be prepared quickly and used easily and instantly, with patient-specific strength adjustments made by the healthcare professional. In the present invention, the anticipated drug-device-patient balance will be provided in optimal level, and transitions from formulation to production, from production to administration to patients will proceed rapidly.

Mucus-removing solutions (mannitol, hypertonic solutions) can also be applied locally (before and/or during and/or after the application of the active substance) together with the therapeutic agents (active substances), and by this means, a much faster recovery process will be possible for the patients, thereby reducing the bad occupancy rate, and/or patient density in hospitals.

In the present invention, the pharmaceutical composition containing favipiravir, mannitol, hydroxychloroquine, umifenovir, molnupiravir, pimodivir, and/or remdesivir, and/or pharmaceutical derivatives thereof may be arranged such that it is for single use or reusable. Single use dosage form is advantageous in the treatment of viral lung diseases since it does not carry the risk of contamination and does not require to add additional excipients (antioxidant, antimicrobial, etc.) to the formulation in order to provide stability. However, the multi-dose form is more advantageous in long term treatments when considering the patient compliance and cost since the patient use the drug at home by himself/herself.

In the present invention administering favipiravir, mannitol, hydroxychloroquine, umifenovir, molnupiravir, pimodivir, and/or remdesivir, and/or pharmaceutical derivatives thereof with a soft mist inhaler having a dosage-adjusting syringe enables that the dosage adjustment for the administration that targets the lung may be performed by the physician in the most sensitive way in response to the requirements of the patient. Said syringe system makes the implementation of patient-specific dosing by physicians significantly more practical in hospitals. In addition, the present syringes that are ready to use and containing favipiravir, mannitol, hydroxychloroquine, umifenovir, molnupiravir, pimodivir, and/or remdesivir, and/or pharmaceutical derivatives thereof can be directly attached to the soft mist inhaler so that the treatment can be offered to the patients quickly in case it is required, thereby eliminating the supply problem. In addition, favipiravir, mannitol, hydroxychloroquine, umifenovir, molnupiravir, pimodivir, and/or remdesivir, and/or pharmaceutical derivatives thereof can be pre-filled into the soft mist inhaler during the production process in the pharmaceutical factory in compliance with the single use or multi-dose use and rendered ready to use by packaging. The present invention also eliminates the bioavailability decrease caused by this administration method, thereby providing these patients with an effective treatment opportunity.

### DESCRIPTION OF THE FIGURES

FIG. 1 illustrates the exploded view of the soft mist inhaler that is used for the administration subject to the invention.

FIG. 2 illustrates the schematic view of the passive vibrating mesh nebulizer that is used for the administration subject to the invention.

FIG. 3 illustrates the schematic view of the active vibrating mesh nebulizer that is used for the administration subject to the invention.

FIG. 4 illustrates the histogram of the study of favipiravir solubility.

FIG. 5 illustrates the phase solubility curve of favipiravir in aqueous solution of HP-β cyclodextrin complex at different concentrations.

FIG. 6 illustrates the data chart showing the stability of solution (5° C.±3° C., 25° C.±2° C./60% RH±5% RH and 40° C.±2° C./75% RH±5% RH and under continuous light) of favipiravirin HP-β cyclodextrin complex in physiological saline solution (PSS), half-physiological saline solution (½ SF) and phosphate buffer (PBS).

FIG. 7 illustrates the data chart showing the stability of solution (5° C.±3° C., 25° C.±2° C./60% RH±5% RH ve 40° C.±2° C./75% RH±5% RH and under continuous light) of favipiravirin in physiological saline solution (PSS), half-physiological saline solution (½ PSS) and phosphate buffer (PBS).

FIG. 8 illustrates a view of the use of a soft mist inhaler, which is used for the administration subject to the invention, with respidrive.

FIG. 9 illustrates a view of the use of a soft mist inhaler, which is used for the administration subject to the invention, with respidrive.

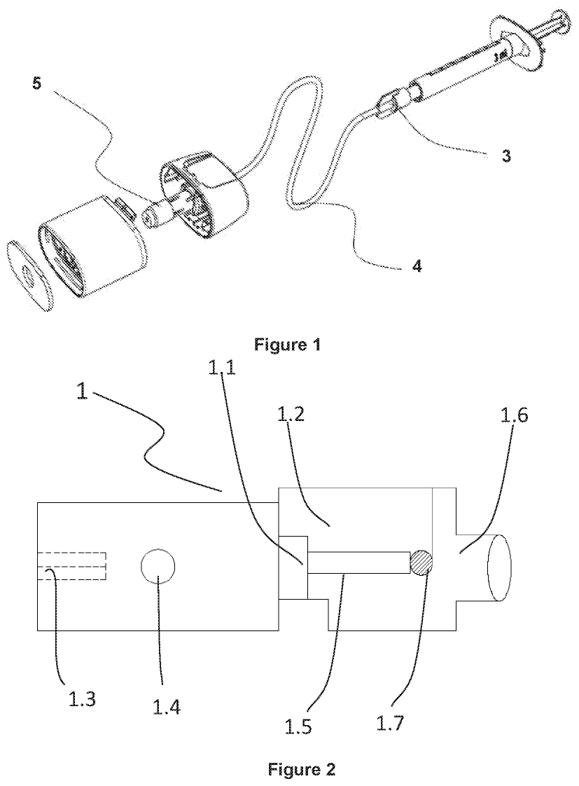

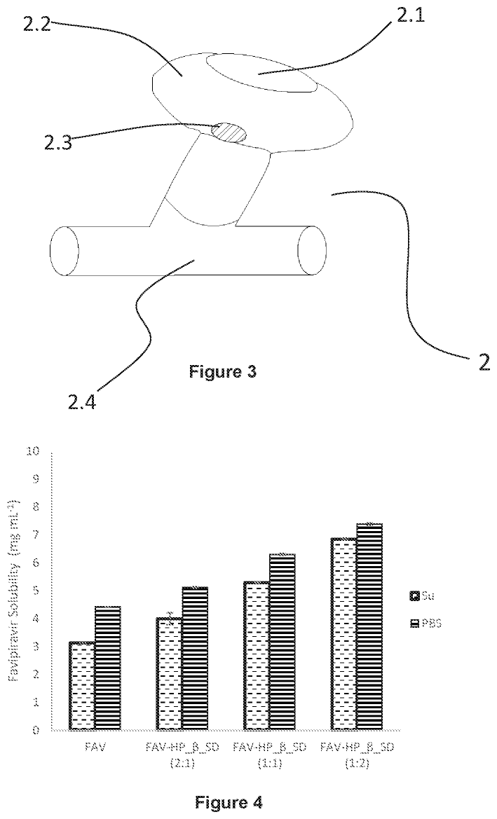

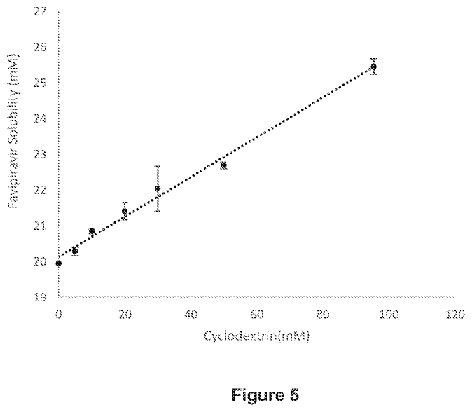

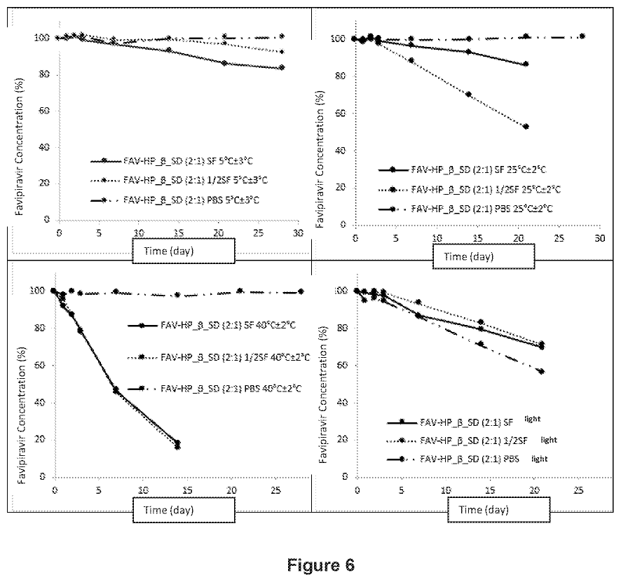

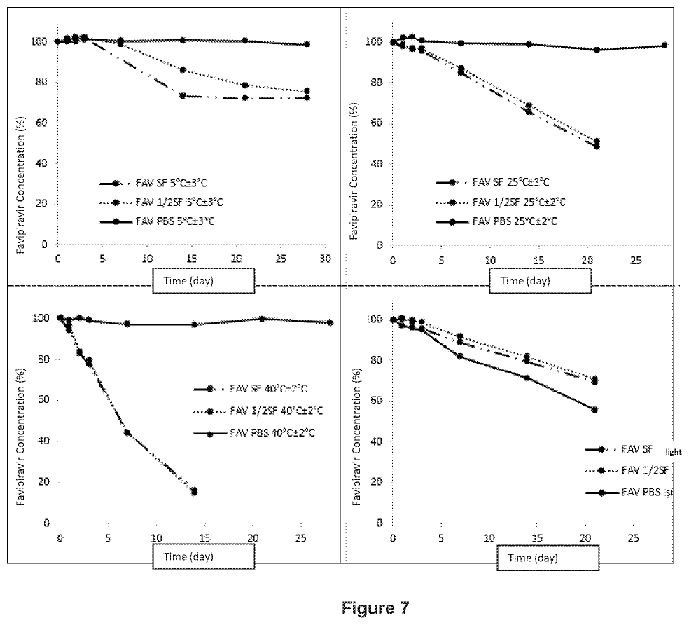

### DESCRIPTION OF ELEMENTS/PARTS/COMPONENTS OF THE INVENTION

The parts and components in the figures are enumerated for a better explanation of the present invention, and correspondence of every number is given below:
1—Passive Vibrating Mesh Nebulizer Device
   1.1—Piezoelectric Crystal
   1.2—Reservoir 1
   1.3—Batteries
   1.4—Operating Button
   1.5—Horn Converter
   1.6—Mouthpiece
   1.7—Mesh 1
2—Active Vibrating Mesh Nebulizer Device
   2.1—Cover
   2.2—Reservoir 2
   2.3—Mesh 2
   2.4—T-shaped Mouthpiece
3—Syringe/injector
4—Connection Tube
5—Soft Mist Inhalation Body
6—Respidrive

### DETAILED DESCRIPTION OF THE INVENTION

The present invention relates to the administration of favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms obtained by water-solubility increasing methods in the treatment of especially COVID-19 and viral lung diseases by means of using soft mist inhaler or vibrating mesh technology (VMT) nebulizer through inhalation route, and the pharmaceutical composition and dosage form thereof for this use. The localization of the drug in the lungs (pharmaceutical composition therein) is 20% and above by means of the use of the pharmaceutical composition of the present invention via soft mist inhaler or vibrating mesh technology (VMT) nebulizer through inhalation. In an embodiment of the present invention, the localization of the drug in the lungs is 40%, 50%, or 60% by means of the use of soft mist inhaler through inhalation. One of the reasons for selecting the active substances of favipiravir, umifenovir, molnupiravir, pimodivir, remdesivir, mannitol, and/or hydroxychloroquine is that they are suitable for local administration to the lung. At least one active substance with antiviral property is used in the present invention, and these are selected among favipiravir, umifenovir, molnupiravir, pimodivir, remdesivir, mannitol, and/or hydroxychloroquine. Here, hydroxychloroquine also has an anti-inflammatory effect. In certain embodiments of the present invention, in addition to at least one antiviral active substance, mannitol and/or hypertonic solution with mucolytic activity may also be used.

Favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, and/or pharmaceutically acceptable derivatives thereof treat the acute lung damage caused by the SARS-CoV-2 virus in the lungs with its antiviral (favipiravir, umifenovir, molnupiravir, pimodivir, and/or remdesivir), anti-inflammatory (hydroxychloroquine), and/or mucolytic properties (mannitol and hypertonic solution). The active substance derivatives mentioned in the pharmaceutical composition of the present invention can be all of the pharmaceutically acceptable derivatives. Examples of pharmaceutically acceptable derivatives may include salts, esters, ethers, bases, solvates, hydrates, or prodrug forms thereof. All derivatives of favipiravir, mannitol, hydroxychloroquine, umifenovir, molnupiravir, pimodivir, and/or remdesivir, which are administered by inhalation in order to target the lungs, are suitable for being locally administered to lungs through the inhalation route by means of using a soft mist inhaler or a passive VMT nebulizer in the treatment of viral lung diseases, especially COVID-19.

In the present invention, favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, and/or pharmaceutically acceptable derivatives thereof may be added into a soft mist inhaler device or a vibrating mesh technology (VMT) nebulizer device at the production stage, or the solution that contains the active substance is packaged and stored in a dropper, prefilled syringe (PFS), ampoule, or vial, and said solution can be added into the device afterward, by patient or healthcare personnel before use in the hospital, or any environment. Therefore, drugs can be delivered in the form of a deposit-solution without requiring production in different strengths at pharmaceutical factories during production. Thus, the application is performed much faster in logistics and hospital with respect to current situation. The convenience to be provided in the area of logistics and dosing by means of the present invention will ensure great speed and efficiency to the health system under pandemic conditions. The efficiency of the treatment will increase and the density rates in the hospitals will be reduced by means of that the recovery times becomes shorter since it is possible for the physicians to apply the dosing of the antiviral, antimalarial, and/or mucolytic active substances to be used in the treatment on a patient-specific basis.

In an embodiment of the present invention, active or passive vibrating mesh technology (VMT) nebulizer is used as a vibrating mesh technology (VMT) nebulizer. Passive vibrating mesh nebulizer device (1) comprises; piezoelectric crystal (1.1), reservoir 1 (1.2), batteries (1.3), operating button (1.4), horn converter (1.5), mouthpiece (1.6), and mesh 1 (1.7). Active vibrating mesh nebulizer device (2), on the other hand, comprises; cover (2.1), reservoir 2 (2.2), mesh 2 (2.3), and t-shaped mouthpiece (2.4). The key component is a mesh plate (1.7), which contains a membrane perforated with precisely created holes. A piezoelectric crystal (1.1) vibrates the mesh of aperture, which is acting as a micropump that draws fluid through the holes in order to create consistently sized fine particles with a diameter of 1-6 μm. The above-mentioned particle size is advantageous since particles with a diameter of 6-10 μm do not move beyond the larger lung airways. VMT Nebulizers produce a low velocity aerosol that minimizes its accumulation (condensation of drug-containing solutions) in the environment and in the upper respiratory tract, thereby optimizing the drug accumulation. They do not generate heat, and therefore, they do not affect the stability of the drug.

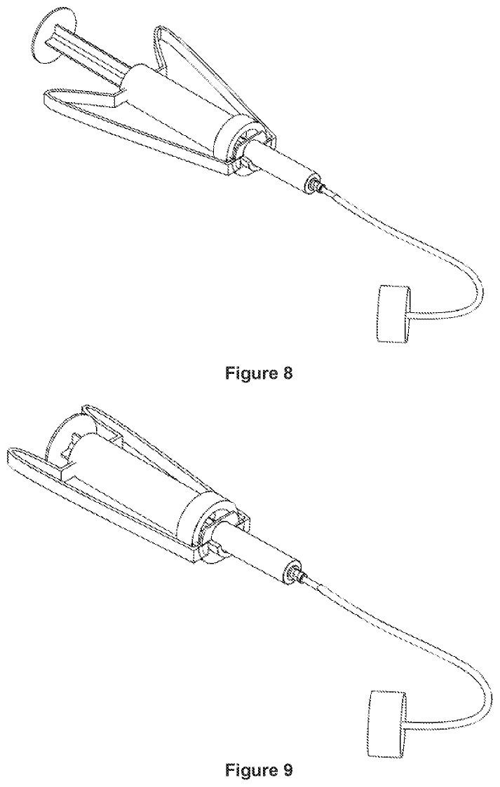

In an embodiment of the present invention favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, and/or pharmaceutically acceptable derivatives thereof are used with soft mist inhaler through inhalation route for the treatment of viral lung diseases, especially COVID-19. In the present invention, PulmoSpray® device available in the state of the art may be used as a soft mist inhaler. The soft mist inhaler comprises a soft mist inhalation body (5) including a special membrane therein, a connecting tube, a syringe, and optionally, in case the respidrive is in the prefilled form, a respidrive (6), or a similar holding system (FIG. 8-9), into which the syringe will be placed; and the soft mist inhalation body (5) provides maximum efficacy for the application. Here, what is meant by maximum efficacy is observing the balance between the highest active substance transfer and the lowest risk of infection. Aerosol droplets suitable for targeting the drug to the lungs are formed when the liquid passes through the membrane in the soft mist inhalation body (5) by means of pushing the liquid with pressure. Said soft mist inhaler is extremely suitable for safety in the use of COVID-19 treatment since it is fitted into the mouth of the patient as a closed system due to its mechanism, and the patient inhales the medicine from the device and exhales through the nose. The droplet size range of the soft mist inhaler, which is effective in the treatment of both COVID-19 and other viral lung diseases, is quite narrow due to the drug/active substance accumulation and ease of use provided by the soft mist inhaler in the lungs. The soft mist inhaler is fitted in the mouth with the mouthpiece, it is inhaled and exhaled through the nose; thus, closed circuit respiration minimizes the environmental contamination of saliva. In addition, the soft mist inhaler has two more advantages that nebulizers do not have: dose accuracy and its practical use. In the soft mist inhaler, dose adjustment depending on weight and age may be performed easily by the physician in the hospital, with patient-specific flexibility by means of the syringe system attached to the device. The parenteral forms of favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, or pharmaceutically acceptable derivatives thereof, which are commercially available as a prefilled syringe may also be used immediately in the patients via use method of the present invention without requiring an additional formulation step due to the fact that the device works with a syringe system.

In the present invention in order to administer favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, and/or pharmaceutically acceptable derivatives thereof, there is a syringe (injector) with dosing function in the soft mist inhaler, which is used for the treatment of viral lung diseases, especially COVID-19. The dosage adjustment for the application that targets the lung may be performed by the physician in the most sensitive way in response to the requirements of the patient by means of said special syringe. Said syringe system makes the implementation of patient-specific dosing by physicians significantly more practical in hospitals. In addition, the parenteral dosage form of the active substances mentioned in the invention, which is available in the form of a ready-to-use syringe, may be directly connected to the soft mist inhaler used in the present invention. The fact that the antiviral, antimalarial and/or mucolytic active substances mentioned in the invention is directly compatible with the device that enables the “formulation-device-administration” triangle to operate in the most efficient way, and the fastest application to the patients, especially to the elderly in the risk group (>65 years) in this pandemic conditions competing with time. Pharmaceutical composition comprisig favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, and/or pharmaceutically acceptable derivatives thereof pass through the inter-device connection tube (4) after the syringe (3), and favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, and/or pharmaceutically acceptable derivatives thereof become the aerosol droplets in the particle size range that may be localized in the lungs, and thus, they can be administered to the lungs via soft mist inhaler by means of the nozzle mechanism in the soft mist inhalation body (5). The soft mist inhaler works with an active mechanism that does not require propellant; the energy required for aerosol production is provided from the inhaler itself, and thus, it is independent of the respiratory capacity of the patient. The size range of the aerosol droplets released from the device is in the range of 1-7 micrometers, and said aerosol droplets are targeted to the lungs. Therefore, the present invention allows for an efficient treatment. Another advantage of the soft mist inhaler is that dosing is performed by means of a syringe.

In an embodiment of the present invention mannitol, favipiravir, hydroxychloroquine, and/or umifenovir are used by means of a soft mist inhaler through inhalation route in the treatment of viral lung diseases, especially COVID-19. Said active substances can be used individually or in combination, and may contain excipients.

In another embodiment of the present invention, favipiravir, molnupiravir, and/or pimodivir, and/or their water-soluble salt forms, and/or their water-soluble cyclodextrin complexes, and/or their water-soluble forms are used in order to administer them by means of soft mist inhaler for the treatment of viral lung diseases, especially COVID-19. Said active substances can be used individually or in combination, and may contain excipients. Beta-cyclodextrin, hydroxypropyl beta cyclodextrin, sulfobutyl ether beta cyclodextrin, alpha cyclodextrin, gamma cyclodextrin, and/or salts thereof included in cyclodextrin derivatives classified as hydrophilic, hydrophobic and nonionic cyclodextrin may be used in the formulation of the present invention.

In another embodiment of the present invention, remdesivir and/or mannitol are used in order to administer them by means of soft mist inhaler for the treatment of viral lung diseases, especially COVID-19. Said active substances can be used individually or in combination, and may contain excipients.

In another embodiment of the present invention, favipiravir, mannitol, hydroxychloroquine, umifenovir, molnupiravir, pimodivir and/or remdesivir, and/or their water-soluble salt forms, and/or their water-soluble cyclodextrin complexes, and/or their water-soluble forms are used in order to administer them by means of active vibrating mesh technology (VMT) nebulizer or passive vibrating mesh technology (VMT) nebulizer for the treatment of viral lung diseases, especially COVID-19. Said active substances can be used individually or in combination, and may contain excipients.

In an embodiment of the present invention, pharmaceutical composition, which is administered through inhalation route comprises; favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, and/or pharmaceutically acceptable derivatives thereof, and a carrier solution that displays solvent properties for said active substances. The pharmaceutical composition to be inhaled comprises favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, and/or pharmaceutically acceptable derivatives thereof, which are dissolved in carrier solution (preferably water for injection). The solvent may be aqueous or non-aqueous within the subject-matter of pharmaceutical composition. A dosage form may be formulated with one or a mixture of more than one pharmaceutically acceptable solvent and can be, but not limited to, glycerol, propylene glycol, polyethylene glycol, polypropylene glycol, ethyl alcohol, isopropyl alcohol, water, mineral oil, peanut oil, and corn oil. The pharmaceutical solvents may be used to prepare the formulation concentrate as well as used for reconstitution of the dosage form. Pharmaceutically acceptable solvents such as water, ethyl alcohol, isopropyl alcohol are evaporable and are usually used to dissolve or disperse the medicament and excipients in the formulation concentrate. Glycerol, propylene glycol and polyethylene glycol are co-solvents and are used to assist in solubilization of water insoluble or poorly water soluble medicaments in the formulation concentrate. Pharmaceutically acceptable reconstituting solvents such as sterile water for injection, water for inhalation, sterile normal saline solution (0.9% NaCl), sterile half saline solution (0.45% NaCl), sterile phosphate buffer solution (pH 4.5-7.4) and/or sterile 5% dextrose solution are used for reconstitution of the dosage form to form a solution or a fine particle suspension of pharmaceutically active substance prior to oral or nasal inhalation via VMT nebulizer or soft mist inhaler.

Pharmaceutical composition of the present invention may be water for injection, or water for inhalation, or physiological saline, or half physiological saline, or sterile inhaled solution in phosphate buffer, containing favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, and/or pharmaceutically acceptable derivatives thereof.

The carrier solution in the composition is used up to the required volume (ml) in order to obtain the solution containing 0.01-20 mg, preferably 0.01-10 mg of favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, and/or pharmaceutically acceptable derivatives thereof; wherein the carrier solution acts as both carrier and solvent and is selected among water for injection, water for inhalation, physiological saline (0.9% NaCl), or half physiological saline (0.45% NaCl), or phosphate buffer (pH 7.4). The solution containing 0.01-20 mg, preferably 0.01-10 mg of active substance is packaged and used as a one-time administration dose. However, in case it is desired to be used in pediatric patient groups, the dose adjustment of the user is performed over said one-time dose. In a preferred embodiment of the present invention, the amount of active substance is 1-10 mg, particularly 1-5 mg. In the present invention, the volume of carrier solution may vary in the range of 1-10 mL depending on the amount of active substance to be used. In the present invention, favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, or pharmaceutically acceptable derivatives thereof are in dissolved form in the 1-10 mL of carrier solution. The volume of carrier solution in the pharmaceutical composition is preferably 1 mL, 2 mL, 5 mL, or 10 mL. In a preferred embodiment of the present invention, the active substance concentration is 1-10 mg/mL, more particularly 1-5 mg/mL. The treatment of viral lung diseases including COVID-19 is provided effectively when the local inhalation is applied with said active substance concentrations. The amount of drug that is administered is low and the side effects thereof is less since the localization rate of said active substances in the lungs is much higher than the conventional dosage forms and other inhaled dosage forms (MDI, DPI, and nebulizers). The risk of contamination will be minimal since the soft mist inhaler and VMT nebulizers to be used as an administration device operate in a closed system. The only administration route of the final composition is through inhalation, however, targeting of local or systemic effect may vary according to the disease that desired to be treated.

In today's antiviral diseases, single doses of favipiravir is in the range of 1-4000 mg. The total daily dose, on the other hand, is currently used in the treatment of COVID-19 in doses up to 7200 mg (This dose may change in the future). The final composition prepared in an embodiment of the present invention is a sterile inhaled solution in a 2 mg/mL concentration, in which said solution is obtained by dissolving 2 mg of favipiravir in 1 mL of phosphate buffer. The aforementioned 2 mg/ml concentration product is packaged as a one-time administration dose. However, in case it is desired to be used in pediatric patient groups, the dose adjustment of the user may be performed over said one-time dose. The administration device used here is a soft mist inhaler (PulmoSpray®), which always provides effective treatment as an inhaler, or vibrating mesh technology (VMT) nebulizer.

In an embodiment of the present invention:
active substance concentration is 1-10 mg/ml
Osmolarity of the formulation is adjusted such that: it is in the range of 250-450 mOsm/L (approximately equivalent to mOsm/kg).
Phosphate buffer (pH 7.4) up to the required ml to obtain favipiravir at the concentration of 1-5 mg/ml.
Physiological saline (0.9% NaCl): up to required ml to obtain favipiravir at the concentration of 1-5 mg/ml.
Half physiological saline (0.45% NaCl): up to required ml to obtain favipiravir at the concentration of 1-5 mg/ml.
HP-β, or SBE-β-CD, or other types of cyclodextrin: is at the molar ratio of 1-1 or 2-1 depending on the amount of favipiravir and solvent used.

In the present invention, HP-β or SBE-β-CD type is used as cyclodextrin. However, alternatively, any other cyclodextrins may be used; for example, α-CD, or β-CD, or γ-CD, or RM-β-CD type of cyclodextrin can be used in the composition of the present invention. In the present invention, cyclodextrin or another solubility enhancer is used in case favipiravir at a dose over 2 mg/mL is used.

An embodiment of the present invention comprises 2 mg/ml of favipiravir, and/or their water-soluble salt forms, and/or their water-soluble cyclodextrin complexes, and/or their water-soluble forms, or pharmaceutically acceptable derivatives thereof, and at least one of the excipients. Said excipients can be selected from the excipients below;
Phosphate buffer (pH 7.4) (as buffer, solvent, carrier)
α-CD, or β-CD, or γ-CD, or particularly HP-β-CD, or SBE-β-CD, or RM-β-CD type of cyclodextrins (as stability enhancers, solubility enhancers, complexing agents)
Physiological saline or half physiological saline (0.9% NaCl) (as solubility enhancer and carrier)
Antioxidants (oxidation inhibitor in single or multiple formulations protects the formulation from free radicals), especially EDTA (Ethylenediaminetetraacetic acid).

In an embodiment of the present invention; each dose contains sterile favipiravir solution, solely or in combination with the cyclodextrin types at a concentration of 2 mg/ml in phosphate buffer (pH 7.4).

The pharmaceutical composition of the present invention, in addition to favipiravir mannitol, hydroxychloroquine, umifenovir, molnupiravir, pimodivir, and/or remdesivir, and/or their water-soluble salt forms, and/or their water-soluble cyclodextrin complexes, and/or their water-soluble forms, or pharmaceutically acceptable derivatives thereof, may also comprise at least one different active substance or at least one excipient. In an embodiment of the present invention, in addition to at least one antiviral active substance, mannitol active substance may be used in the pharmaceutical composition.

In an embodiment of the present invention, in the pharmaceutical composition, mannitol may be added to the solution containing favipiravir, hydroxychloroquine, umifenovir, molnupiravir, pimodivir, and/or remdesivir, and/or their water-soluble salt forms, and/or their water-soluble cyclodextrin complexes, and/or their water-soluble forms, or pharmaceutically acceptable derivatives thereof. Thus, also the opening effect of the mucus plug in the lungs is provided.

In said pharmaceutical composition, in addition to favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, and/or pharmaceutically acceptable derivatives thereof, in case of using a different active substance or directly in addition to said pharmaceutical composition, excipient(s) may be used. The subject-matter of pharmaceutical composition can contain at least one excipient selected from tonicity adjusting excipients, pH adjusting or buffering agents, tonicity adjusting agents, antioxidants, antimicrobial preservatives, surfactants, solubility enhancers (co-solvents), stabilizing agents, excipients for sustained release or prolonged local retention, wetting agents, dispensing agents, taste-masking agents, sweeteners, and/or flavours. These excipients are used to obtain an optimal pH, viscosity, surface tension and taste, which support the formulation stability, the aerosolization, the tolerability, and/or the efficacy of the formulation upon inhalation.

One or more co-solvents (solubility enhancer) may be included into the subject-matter of pharmaceutical composition to aid the solubility of the active substance and/or other excipients. Examples of pharmaceutically acceptable co-solvents include propylene glycol, dipropylene glycol, ethylene glycol, glycerol, ethanol, polyethylene glycols (for example PEG300 or PEG400), methanol, polyethylene glycol castor oil, polyoxyethylene castor oil, and/or lecithin. As co-solvents; alcohols (ethanol, isopropyl alcohol, etc.), glycols (propylene glycol, polyethylene glycol, polypropylene glycol, etc.) can also be used.

Stabilizing agents which can be used for the subject-matter of phrmaceutical composition are antioxidant and chelating agents that are capable of inhibiting oxidation reaction and chelating metals, respectively, to improve stability of pharmaceutically active ingredient and excipients. Dosage forms may be formulated with one or more pharmaceutically acceptable stabilizing agents at a concentration suitable for the intended pharmaceutical applications, and may be, but not limited to, chelating agents such as disodium edetate (Ethylenediaminetetraacetic acid, EDTA) or its sodium salt, citric acid, sodium citrate, vitamin E, ascorbic acid, ascorbyl palmitate, butylated hydroxyanisole, butylated hydroxytoluene, hypophosphorous acid, monothioglycerol, propyl galate, sodium bisulfite, sodium metabisulfite, sodium formaldehyde sulfoxylate, thiourea, lysine, tryptophan, phenylpropyl glycine, glycine, glutamic acid, leucine, isoleucine, serine, tea polyphenols, ascorbyl palmitate, hydroxymethyl ester, hydroxyethyl tetramethyl piperidinol, bis (2,2,6,6-tetramethyl-4-piperidyl) sebacate, polysuccinate (4-hydroxy-2,2,6,6-tetramethyl-1-piperidinylethanol) ester, 2-[2-hydroxy-4-[3-(2-ethylhexyloxy)-2-hydroxypropoxy]phenyl]-4,6-bis (2,4-dimethylphenyl) and/or 1,3,5-triazine.

Antioxidants, which are natural or synthetic substances which prevent or interrupt the oxidation of active agents and/or oxidative injury in stressed tissues and cells, can be used in the subject-matter of pharmaceutical composition. Antioxidants which can be used in the subject-matter of pharmaceutical composition can be adjuvants which are oxidisable themselves (i.e. primary antioxidants) or adjuvants that act as reducing agents (i.e. reducing antioxidants), such as tocopherol acetate, lycopene, reduced glutathione, catalase and/or peroxide dismutase. Other adjuvants used to prevent oxidative reactions are synergistic antioxidants, which do not directly act in oxidation processes, but indirectly via the complexation of metal ions that are known to catalyse oxidation reactions. Frequently used synergistic antioxidants are ethylenediamine tetraacetic acid (EDTA) and its derivatives. Further useful antioxidants (primary, reducing and/or synergistic anti-oxidising working mechanism) are ascorbic acid and/or its salts, esters of ascorbic acid, fumaric acid and/or its salts, malic acid and/or its salts, citric acid and/or its salts, butyl hydroxy anisole, butyl hydroxy toluene, propyl gallate and/or maltol. As an alternative to generally used antioxidants, substances such as acetylcysteine, R-cysteine, vitamin E TPGS, pyruvic acid and/or its magnesium and/or sodium salts, gluconic acid and/or its magnesium and/or sodium salts, might also be useful in formulations for inhalation. The salts of gluconic acid have the additional advantage that they have been described to have an anti-oxidising effect on stressed tissues and cells, which can be particularly advantageous in the treatment of inflammations, as oxygen radicals induce and perpetuate inflammatory processes. Also pyruvate salts are believed to have such in vivo anti-oxidising effects. An additional measure to prevent oxidation and to contribute to the prevention of the undesired discolouration is the replacement of oxygen above the solution by an inert gas but not limited to such as nitrogen or argon.

Antimicrobial preservatives can be used in the subject-matter of pharmaceutical composition to inhibit the growth of microorganisms. Dosage forms may be formulated with one or more pharmaceutically acceptable antimicrobial preservatives at suitable concentrations to prevent microbial growth. Compositions for administration to the lungs or nose may contain one or more excipients, may be protected from microbial or fungal contamination or growth by the inclusion of one or more preservatives. Examples of pharmaceutically acceptable antimicrobial agents or preservatives include, but are not limited to, quaternary ammonium compounds (e.g., benzalkonium chloride, benzethonium chloride, cetrimide, cetylpyridinium chloride, lauralconium chloride and/or myristyl picolinium mercuric chloride), thimerosal alcoholic agents (e.g. chlorobutanol, phenylethyl alcohol and/or benzyl alcohol), antibacterial esters (e.g. parahydroxybenzoic acid esters), chelating agents such as disodium edetate (EDTA) other antimicrobial agents such as chlorhexidine, chlorocresol, sorbic acid and/or its salts (such as potassium sorbate) and polymyxin. Examples of pharmaceutically acceptable antifungal agents or preservatives include, but are not limited to, sodium benzoate, sorbic acid, sodium propionate, methylparaben, ethylparaben, propylparaben, butylparaben, ethyl p-hydroxybenzoate and/or n-propyl p-hydroxybenzoate. Benzalconium chloride, benzoic acid, benzoates (e.g sodium benzoate) can also be used as antimicrobials.

pH adjusting or buffering agents can be used in the subject-matter of pharmaceutical composition to adjust or maintain the pH of pharmaceutical dosage form to a desired range for the following reasons: to provide an environment for better product stability that pharmaceutical active ingredient may express a better chemical stability within a certain pH range, or to provide better comfort for the patient at administration. Extreme pH may create irritation and/or discomfort to the site of administration, and to provide a pH range for better antimicrobial preservative activity. The subject-matter of pharmaceutical composition can comprise one or more excipients to adjust and/or buffer the pH value of the solution. For adjusting and optionally buffering pH, physiologically acceptable acids, bases, salts, and/or combinations thereof may be used. Excipients often used for lowering the pH value or for application as acidic component in a buffer system are strong mineral acids, in particular sulfuric acid and hydrochloric acid. Also inorganic and organic acids of medium strength as well as acidic salts may be used such as phosphoric acid, citric acid, tartaric acid, succinic acid, fumaric acid, methionine, acidic hydrogen phosphates with sodium or potassium, lactic acid, and/or glucuronic acid. Excipients suitable for raising the pH or as basic component in a buffer system are, in particular, mineral bases such as sodium hydroxide or other alkaline earth hydroxides and oxides such as magnesium hydroxide and calcium hydroxide, ammonium hydroxide and basic ammonium salts such as ammonium acetate, as well as basic amino acids such as lysine, carbonates such as sodium or magnesium carbonate, sodium hydrogen carbonate, and citrates such as sodium citrate. The subject-matter of pharmaceutical composition can comprise a buffer system consisting of two components. One of the most preferred buffer systems contains citric acid-sodium citrate, citric acid-phosphoric acid disodium hydrogen, potassium dihydrogen phosphate-disodium hydrogen phosphate or citric acid-sodium hydroxide, trometamol, disodium phosphate (for example dodecahydrate, heptahydrate, dihydrate and anhydrous forms thereof) and/or sodium mixtures. Nevertheless, other buffering systems may also be used.

A tonicity adjusting agent is one or more pharmaceutical excipients which are osmotically active and which are used in common practice for the purpose of adjusting the osmolality or tonicity of liquid pharmaceutical formulations. Mainly tonicity adjusting agents are used to enhance the overall comfort to the patient upon administration. A tonicity adjusting agent can be used in the subject-matter of pharmaceutical composition selected from sodium chloride, mannitol or dextrose. Other salts that can be used in the subject-matter of pharmaceutical composition for adjusting tonicity are sodium gluconate, sodium pyruvate and/or potassium chloride. Also carbohydrates can be used for this purpose. Examples are sugars such as glucose, lactose, sucrose or trehalose, sugar alcohols such as xylitol, sorbitol and/or isomaltol. Alternately, the dosage form may be formulated without the addition of a major tonicity adjusting agent. The desired tonicity of the dosage form is achieved by reconstituting with a sterile isotonic saline solution.

In the present invention; organic acids as pH adjusting agents; ascorbic acid, citric acid, malic acid, tartaric acid, maleic acid, succinic acid, fumaric acid, acetic acid, formic acid, and/or propionic acid; or as inorganic acid, hydrochloric acid and/or sulfuric acid can be used. In the present invention, organic bases such as alkali metal hydroxides or alkali metal carbonates can be used. In the composition according to the present invention, phosphate buffer or citrate buffer can be used as a buffering agent. One or more tonicity adjusting agents may be added into said composition in order to provide the desired ionic strength. In the composition of the present invention, both organic and inorganic tonicity adjusting agent may be used; these can be sodium chloride and dextrose.

The surface tension of a liquid composition is important for optimal inhalation. Compositions with a desirable surface tension are expected to show a good spreadability on the mucous membranes of the respiratory tract. In order to enable the formulation to be atomized smoothly and form uniform and stable aerosol particles to be absorbed by the patient, an optimal surface tension needed. Furthermore, the surface tension might need to be adjusted to allow a good emptying of the composition from its primary package. Surfactants are materials with at least one relatively hydrophilic and at least one relatively lipophilic molecular region that accumulate at hydrophilic-lipophilic phase interfaces and reduce the surface tension. The surface-active materials can be ionic or non-ionic. Particularly preferred surfactants are those that have a good physiological compatibility and that are considered safe for oral or nasal inhalation. Preferred surfactant in the subject-matter of phrmaceutical composition can be tyloxapol, polysorbates, polysorbate 20, polysorbate 60, polysorbate 80, lecithin, vitamin F TPGS, macrogol hydroxystearates and/or macrogol-15-hydroxystearate. The surfactant used in the subject-matter of phrmaceutical composition might also comprise a mixture of two or more surfactants, such as polysorbate 80 in combination with vitamin E TPGS.

In the present invention, non-ionic surfactants, anionic surfactants, cationic surfactants, or zwitterionic surfactants can be surfactants. Here, preferably it can be selected from one or more surfactants or more non-ionic surfactants. Polyoxyethylene glycol sorbitan alkyl esters such as polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), polysorbate 80 (polyoxyethylene (20) sorbitan monooleate); or sorbitan alkyl esters such as sorbitan monolaurate, sorbitan monopalmitate, sorbitan monostearate, sorbitan tristearate, or sorbitan monooleate can be used as surfactant in the composition according to the present invention.

In some of the embodiments of the invention, also taste-masking agents or sweetening agents or flavoring agents can be used as excipient. A bad taste of formulations for inhalation is extremely unpleasant and irritating. The bad taste sensation upon inhalation results from direct deposition of aerosol droplets in the oral and pharyngeal region upon oral inhalation, from transport of drug from the nose to the mouth upon nasal inhalation, and from transport of the drug from the respiratory tract to the mouth related to the mucociliary clearance in the respiratory system. A taste-masking agent is any pharmaceutically acceptable compound or mixture of compounds capable of improving the taste of an aqueous system, regardless of the mechanism by which the improvement is brought about. For example, the taste-masking agent may cover the poor taste, i.e. reduce the intensity by which it is perceived, or it may correct the taste by adding another, typically more pleasant, flavour to the composition, thereby improving the total organoleptic impression. Other taste-masking mechanisms are complexation, encapsulation, embedding or any other interaction between drug and other compounds of the composition. Taste-masking agent which can be used in the subject-matter of phrmaceutical composition is selected from the group of pharmaceutically acceptable sweeteners such as saccharin, aspartame, cyclamate, sucralose, acesulfame, neotame, thaumatin, and/or neohesperidine, including salts and solvates thereof such as the sodium salt of saccharin and the potassium salt of acesulfame. Furthermore, sugars such as sucrose, trehalose, fructose, and lactose, or sugar alcohols such as xylitol, mannitol or isomalt can be used. Further useful taste-masking agents include pharmaceutically acceptable surfactants, alkaline earth metal salts, organic acids such as citric acid and lactic acid, and/or amino acids such as arginine. Also aromatic flavours, such as the ingredients of essential oils (menthol, thymol or cineol) may be used in the subject-matter of phrmaceutical composition to improve the taste and tolerability of the composition according to the invention.

Wetting or dispensing agents can be used in the subject-matter of pharmaceutical composition to increase wettability and assist in dispersing of water insoluble or poorly water soluble particles. For water insoluble and poorly water soluble medicaments, the addition of one or more wetting or dispersing agents to the dosage formulation can help the release of the impregnated pharmaceutical active substance particles from the supporting material into the reconstituted solution and can help the dispersion of the particles to form a fine suspension. Examples of pharmaceutically acceptable wetting and dispersing agents suitable for oral or nasal inhalation for the subject-matter of phrmaceutical composition are poloxamers, oleic acid or its salts, lecithin, hydrogenated lecithin, sorbitan fatty acid esters, oleyl alcohol, phospholipids including but not limited to phosphatidylglycerol, phosphatidylcholine, polyoxyethylene fatty alcohol ethers, polyoxypropylene fatty alcohol ether, polyoxyethylene fatty acid ester, glycerol fatty acid esters, glycolipid such as sphingolipid and sphingomyelin, polyoxyethylene glycol fatty acid ester, polyol fatty acid esters, polyethylene glycol glycerol fatty acid esters, polypropylene glycol fatty acid esters, ethoxylated lanolin derivatives, polyoxyethylene fatty alcohol, polyoxyethylene sorbitan fatty acid esters, polyoxyethylene stearate, propylene glycol alginate, dilauryldimethylammonium chloride, D-a-tocopheryl-PEG 1000 succinate, Polyoxy 40 stearate, polyoxyethylene-polyoxypropylene block copolymers, polyoxyethylene vegetable oils, fatty acid derivatives of amino acids, glyceride derivatives of amino acids, benzalkonium chloride and/or bile acids.

In the present invention, the primary packaging used for the active substances of favipiravir, mannitol, hydroxychloroquine, umifenovir, molnupiravir, pimodivir, and/or remdesivir should be amber-colored or opaque, and it is made of a pharmaceutical-grade material, which is biologically compatible with the content of said pharmaceutical composition. The material of the chamber that will contain the composition of the present invention may be glass or synthetic material. The formulation may be packaged in a single dose or multi-dose form. In an embodiment of the present invention: the composition can be prepared in a double or binary pharmaceutical active dosage form by means of preparing the main antiviral solution as hypertonic and adding/without adding the varying concentration of mannitol. The formulation may be pre-filled to the inhaler or may be in a form that allows the formulation to be provided to the inhaler during use. Unit-dose respiratory drugs are packaged in soft plastic containers, which are generally formed of low-density polyethylene (LDPE) or LPDE in order to control costs and facilitate the opening of containers. In the present invention, the primary packaging to be used for LMWH may be made of glass material.

Said composition may be for single use or reusable. In case said composition is reusable, it may also contain antioxidant agent, antimicrobial preservative, vitamin, pH adjusting agent, buffering agent, surfactant, tonicity adjusting agent, stabilizer, complexing agent. In case it is single use, only carrier solution (water for injection, inhalation water or phosphate buffer, etc.) will be sufficient as an excipient. However, an additional excipient is also used in case of adding a different active substance to the single use composition. In case it is reusable or in combination with other active substances, substances from the excipient groups that are indicated in detail above may be added to the formulation content.

The pharmaceutical composition according to the present invention is prepared in solution form, and it is administered to the patient through inhalation route by means of soft mist inhaler or VMT nebulizer devices. The pharmaceutical composition according to the present invention can be solution. In addition to the pharmaceutical composition according to the present invention, said composition that can be combined with antivirals, mucolytic agents, vitamins or corticosteroids is applied especially in the treatment of COVID-19 and influenza. The patient groups to which the composition according to the invention may be applied are inpatients, outpatients, or home care patients.

Within the scope of the present invention, the indications that favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, and/or pharmaceutically acceptable derivatives thereof in the sterile solution dosage form may be used in the lungs by means of soft mist inhaler or VMT are viral lung diseases, and said compound is particularly indicated in COVID-19 and influenza.

Within the scope of the present invention, the active substance groups according to the invention, which are used for administration by means of soft mist inhaler or VMT nebulizer, are selected in accordance with their characteristics, especially physico-chemical properties thereof such as water solubility, target treatment pathogen, bioavailability ect.

In the present invention, a study for solubility of favipiravir active substance was conducted. The solubility of favipiravir, favipiravir HP-β-cyclodextrin, and SBE-β-cyclodextrin complex, and molar concentrations of 2:1, 1:1 and 1:2 in distilled water and phosphate buffer (pH:7.4) (PBS) were studied. The results are as in table: It was observed that the solubility in PBS further increased in consideration of these results.

TABLE 1
Preformulation Table

| | | | Favipiravir-HP- |
|---|---|---|---|
| | | | β-Cyclodextrin |
| Formula- | | | (molar |
| tion | Favipiravir | | concentration |
| Number | Concentration | Formulation Solvent | ratio) |
| 1 | 2 mg/mL | Physiological saline (0.9% | — |
| | | NaCl) | |
| 2 | 2 mg/mL | Half physiological saline | — |
| | | (0.45% NaCl) | |
| 3 | 2 mg/mL | Phosphate buffer (pH 7.4): | — |
| 4 | 2 mg/mL | Physiological saline (0.9% | 1:1 |
| | | NaCl) | |
| 5 | 2 mg/mL | Half physiological saline | 1:1 |
| | | (0.45% NaCl) | |
| 6 | 2 mg/mL | Phosphate buffer (PBS, pH | 1:1 |
| | | 7.4) | |
| 7 | 2 mg/mL | Physiological saline (0.9% | 2:1 |
| | | NaCl) | |
| 8 | 2 mg/mL | Half physiological saline | 2:1 |
| | | (0.45% NaCl) | |
| 9 | 2 mg/mL | Phosphate buffer (PBS, pH | 2:1 |
| | | 7.4) | |

In the present invention, pH analysis was performed for the composition containing favipiravir. The smallest pH value of favipiravir is 3.56-3.62 in combinations of physiological saline, half physiological saline, and HP-β-cyclodextrin since said favipiravir has an acidic pKa. When it is formulated with phosphate buffer, pH values thereof approach 7,4. Therefore, in consideration of all combinations, the pH interval for the formulations was kept in the range of 3.5-8.0.

In the present invention, osmolarity analysis was performed for the composition containing favipiravir. When the osmolarities of the prepared favipiravir (2 mg/mL) solutions are examined, it is observed that the osmolarity (299 mOsm/Kg) of the favipiravir solution in phosphate buffer (pH 7.4) is significantly similar to the osmolarity (303 mOsm/Kg) of favipiravir in physiological saline. Therefore, literature data support that formulations with said osmolarity values are suitable for administering said formulations directly to the lungs through inhalation [1]. The osmolarity value of the HP-β cyclodectrin complex (2:1 molar ratio) of favipiravir that is prepared in phosphate buffer was found 5360 mOsm/Kg (Table 2).

TABLE 2
PH, viscosity, and osmolarity values of solutions
containing favipiravir active subtance

| | | | Osmolarity |
|---|---|---|---|
| Formulations | pH | Viscocity | (mOsm/Kg) |
| Solution (2 mg/mL) of | 6.50 | 1.009 mPa · s | 299 |
| Favipiravir Active | | | |
| Substance in | | | |
| phosphate buffer (pH: | | | |
| 7.4) | | | |
| Solution of HP- β- | 3.59 | 1.238 mPa · s | 5360 |
| Cyclodextrin complex | | | |
| (2:1) of Favipiravir | | | |
| Active Substance in | | | |
| phosphate buffer | | | |
| (pH: 7.4) | | | |

Normal lung histology that is characterized by the presence of thin alveolar septums, alveolar sacs of different sizes, and well organized alveolar canals was observed as a result of the examination under light microscopy of the lung tissue samples of rats in the physiological saline-administered control group, and the groups, in which is favipiravir with soft mist form is administered. In addition, it was determined that the histological damage scores of the lungs obtained from stained tissue sections of the experimental groups, in which physiological saline and favipiravir are administered for histopathological evaluation (H&E) are not statistically different from each other. It has been proven that administering said compound by means of the soft mist inhaler or VMT of the present invention through inhalation is safe since these preclinical studies indicate that administration of favipiravir does not cause any damage to the lungs.

In a preferred embodiment of the present invention it was observed that a sterile solution of favipiravir (2 mg/mL) in phosphate buffer (pH 7.4) has the most suitable properties for inhalation after the stability studies, in vitro characterization, cellular toxicity, and in vivo studies.

### EXAMPLES

All example formulations are in the range of pH:3.5-8.0, and contain 1-10 mg of favipiravir (preferably contain 1-10 mg/mL of favipiravir).

Example 1: Favipiravir in phosphate buffer

Example 2: Favipiravir+Mannitol in phosphate buffer.

Example 3: Favipiravir+Hypertonic Saline in phosphate buffer.

Example 4: Favipiravir+alpha cyclodextrin (α-CD), or beta cyclodextrin (β-CD), or gamma cyclodextrin (γ-CD), or hydroxypropyl beta cyclodextrin (HP-β-CD), or sulfobutyl ether beta cyclodextrin (SBE-β-CD), or randomized methylated beta cyclodextrin (RM-β-CD) in phosphate buffer.

Example 5: Favipiravir+Mannitol+α-CD, or β-CD, or γ-CD, or HP-β-CD, or SBE-β-CD, or RM-β-CD in phosphate buffer.

Example 6: Favipiravir+Hypertonic saline+ α-CD, or β-CD, or γ-CD, or HP-β-CD, or SBE-β-CD, or RM-β-CD in phosphate buffer.

Example 7: Faviripiravir in physiological saline (0.9% NaCl).

Example 8: Favipiravir+Mannitol in physiological saline (0.9% NaCl).

Example 9: Favipiravir+α-CD, or β-CD, or γ-CD, or HP-β-CD, or SBE-β-CD, or RM-β-CD in physiological saline (0.9% NaCl).

Example 10: Favipiravir+Mannitol+α-CD, or p-CD, or γ-CD, or HP-β-CD, or SBE-β-CD, or RM-β-CD in physiological saline (0.9% NaCl).

Example 11: Favipiravir in half-normal physiological saline (0.45% NaCl).

Example 12: Favipiravir+Mannitol in half-normal physiological saline (0.45% NaCl).

Example 13: Favipiravir+α-CD, or β-CD, or γ-CD, or HP-β-CD, or SBE-β-CD, or RM-β-CD in half-normal physiological saline (0.45% NaCl).

Example 14: Favipiravir+Mannitol+α-CD, or β-CD, or γ-CD, or HP-β-CD, or SBE-β-CD, or RM-β-CD in half-normal physiological saline (0.45% NaCl).

### REFERENCES

1. Portel L, Tunon de Lara J M, Vernejoux J M, Weiss I, Taytard A. Osmolarité des solutions utilisées en nébulisation [Osmolarity of solutions used in nebulization]. Rev Mal Respir. 1998 April; 15(2):191-5. French. PMID: 9608990.

1. A pharmaceutical composition comprising favipiravir, mannitol, hydroxychloroquine, and/or umifenovir active substances, and/or at least one excipient in order to locally administer it to the lungs for use in the treatment of viral lung diseases including COVID-19 disease caused by Severe Acute Respiratory Syndrome-Coronavirus-2 (SARS-CoV-2) by means of soft mist inhaler through inhalation.

2. A pharmaceutical composition comprising favipiravir, molnupiravir, and or pimodivir, and/or their water-soluble salt forms, and/or their water-soluble cyclodextrin complexes, and/or their water-soluble forms, and/or at least one excipient in order to locally administer it to the lungs for use in the treatment of viral lung diseases including COVID-19 disease caused by Severe Acute Respiratory Syndrome-Coronavirus-2 (SARS-CoV-2) by means of soft mist inhaler through inhalation.

3. A pharmaceutical composition comprising remdesivir, and/or mannitol, and/or at least one excipient in order to locally administer it to the lungs for use in the treatment of viral lung diseases including COVID-19 disease caused by Severe Acute Respiratory Syndrome-Coronavirus-2 (SARS-CoV-2) by means of soft mist inhaler through inhalation.

4. A pharmaceutical composition comprising favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms in order to locally administer it to the lungs for use in the treatment of viral lung diseases including COVID-19 disease caused by Severe Acute Respiratory Syndrome-Coronavirus-2 (SARS-CoV-2) by means of active vibrating mesh technology nebulizer or passive vibrating mesh technology nebulizer through inhalation.

5. A pharmaceutical composition comprising favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, or pharmaceutically acceptable derivatives thereof, which are dissolved in a carrier solution, in order to locally administer it to the lungs for use in the treatment of viral lung diseases including COVID-19 disease caused by Severe Acute Respiratory Syndrome-Coronavirus-2 (SARS-CoV-2) by means of soft mist inhaler, or active vibrating mesh technology nebulizer, or passive vibrating mesh technology nebulizer through inhalation.

6. A pharmaceutical composition according to claim 5, characterized in that a pharmaceutically acceptable derivative thereof is selected from salts, esters, ethers, bases, solvates, hydrates, or their forms used as prodrug.

7. A pharmaceutical composition according to claim 5, characterized in that, the carrier solution is water for injection, water for inhalation, physiological saline (0.9% NaCl), half physiological saline (0.45% NaCl), or phosphate buffer (pH 4.5-7.4).

8. A pharmaceutical composition according to any one of the preceding claims, characterized in that, it comprises; 0.01-20 mg of favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, or pharmaceutically acceptable derivatives thereof, which are dissolved in a carrier solution.

9. A pharmaceutical composition according to claim 8, characterized in that, it comprises; 0.01-10 mg of favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, or pharmaceutically acceptable derivatives thereof, which are dissolved in a carrier solution.

10. A pharmaceutical composition according to claim 9, characterized in that, it comprises; 1-10 mg of favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, or pharmaceutically acceptable derivatives thereof, which are dissolved in a carrier solution.

11. A pharmaceutical composition according to claim 10, characterized in that, it comprises; 1-5 mg of favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, or pharmaceutically acceptable derivatives thereof, which are dissolved in a carrier solution.

12. A pharmaceutical composition according to any one of claims 8 to 11, characterized in that, favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, or pharmaceutically acceptable derivatives thereof are in dissolved form in the 1-10 mL of carrier solution.

13. A pharmaceutical composition according to claim 12, characterized in that, the volume of carrier solution is 1 mL, 2 mL, 5 mL, or 10 mL.

14. A pharmaceutical composition according to any one of the preceding claims, characterized in that, it comprises; 1-10 mg/mL of favipiravir, or its water-soluble cyclodextrin complexes, and/or its water-soluble forms, or its pharmaceutically acceptable derivatives, which are dissolved in a carrier solution.

15. A pharmaceutical composition according to claim 14, characterized in that, it comprises; 1-5 mg/mL of favipiravir, or its water-soluble cyclodextrin complexes, and/or its water-soluble forms, or its pharmaceutically acceptable derivatives, which are dissolved in a carrier solution.

16. A pharmaceutical composition according to any one of the preceding claims, characterized in that, it comprises at least one active substance with antiviral property, and/or at least one excipient.

17. A pharmaceutical composition according to claim 16, characterized in that, the active substance with antiviral property is selected from favipiravir, umifenovir, molnupiravir, pimodivir, remdesivir, and/or hydroxychloroquine.

18. A pharmaceutical composition according to any one of the preceding claims, characterized in that, it further comprises at least one different active substance and/or at least one excipient.

19. A pharmaceutical composition according to claims 16 or 18, characterized by comprising at least one excipient selected from tonicity adjusting excipients, pH adjusting agents, buffering agents, tonicity adjusting agents, antioxidants, antimicrobial preservatives, surfactants, solubility enhancers (co-solvents), stabilizing agents, excipients for sustained release or prolonged local retention, wetting agents, dispensing agents, taste-masking agents, sweeteners, and/or flavour.

20. A pharmaceutical composition according to claim 19, characterized in that co-solvent can be selected from propylene glycol, dipropylene glycol, ethylene glycol, glycerol, ethanol, polyethylene glycols, PEG300, PEG400, methanol, polyethylene glycol castor oil, polyoxyethylene castor oil and/or lecithin.

21. A pharmaceutical composition according to claim 19, characterized in that stabilizing agent can be selected from EDTA or its sodium salt, citric acid, sodium citrate, vitamin E, ascorbic acid, ascorbyl palmitate, butylated hydroxyanisole, butylated hydroxytoluene, hypophosphorous acid, monothioglycerol, propyl galate, sodium bisulfite, sodium metabisulfite, sodium formaldehyde sulfoxylate, thiourea, lysine, tryptophan, phenylpropyl glycine, glycine, glutamic acid, leucine, isoleucine, serine, tea polyphenols, ascorbyl palmitate, hydroxymethyl ester, hydroxyethyl tetramethyl piperidinol, bis (2,2,6,6-tetramethyl-4-piperidyl) sebacate, polysuccinate (4-hydroxy-2,2,6,6-tetramethyl-1-piperidinylethanol) ester, 2-[2-hydroxy-4-[3-(2-ethylhexyloxy)-2-hydroxypropoxy] phenyl]-4,6-bis (2,4-dimethylphenyl) and/or 1,3,5-triazine.

22. A pharmaceutical composition according to claim 19, characterized in that antioxidant can be selected from primary antioxidants, reducing antioxidants and/or synergistic antioxidants.

23. A pharmaceutical composition according to claim 19, characterized in that antioxidant can be selected from tocopherol acetate, lycopene, reduced glutathione, catalase, peroxide dismutase, acetylcysteine, R-cysteine, vitamin E TPGS, pyruvic acid and/or its magnesium or sodium salts, gluconic acid and/or its magnesium and/or sodium salts, ethylenediamine tetraacetic acid (EDTA) and/or its derivatives, ascorbic acid, esters of ascorbic acid, fumaric acid, malic acid, citric acid, butyl hydroxy anisole, butyl hydroxy toluene, propyl gallate, maltol and/or salts thereof.

24. A pharmaceutical composition according to claim 19, characterized in that antimicrobial preservative can be selected from quaternary ammonium compounds, thimerosal alcoholic agents, antibacterial esters, chelating agents and/or antifungal agents.

25. A pharmaceutical composition according to claim 19, characterized in that antimicrobial preservative can be selected from benzalkonium chloride, benzethonium chloride, cetrimide, cetylpyridinium chloride, lauralconium chloride, myristyl picolinium mercuric chloride, chlorobutanol, phenylethyl alcohol, benzyl alcohol, parahydroxybenzoic acid esters, disodium edetate (ethylenediaminetetraacetic acid, EDTA), chlorhexidine, chlorocresol, sorbic acid and/or its salts, potassium sorbate, polymyxin, sodium benzoate, sorbic acid, sodium propionate, methylparaben, ethylparaben, propylparaben, butylparaben, ethyl p-hydroxybenzoate and/or n-propyl p-hydroxybenzoate.

26. A pharmaceutical composition according to claim 19, characterized in that, pH adjusting agent can be selected from physiologically acceptable acids, bases, salts or combinations thereof.

27. A pharmaceutical composition according to claim 19, characterized in that, pH adjusting agent can be selected from strong mineral acids, mineral bases, inorganic acids of medium strength, organic acids of medium strength, alkaline earth hydroxides and oxides, basic ammonium salts, carbonates, citrates.

28. A pharmaceutical composition according to claim 19, characterized in that, pH adjusting agent can be selected from sulfuric acid, hydrochloric acid, phosphoric acid, citric acid, tartaric acid, succinic acid, fumaric acid, methionine, acidic hydrogen phosphates with sodium or potassium, lactic acid, glucuronic acid, sodium hydroxide, magnesium hydroxide, calcium hydroxide, ammonium acetate, lysine, sodium carbonate, magnesium carbonate, sodium hydrogen carbonate, sodium citrate.

29. A pharmaceutical composition according to claim 19, characterized in that, buffering agent can be selected from citric acid-sodium citrate, citric acid-phosphoric acid disodium hydrogen, potassium dihydrogen phosphate-disodium hydrogen phosphate, citric acid-sodium hydroxide, trometamol, disodium phosphate, dodecahydrate, heptahydrate, dihydrate and anhydrous forms thereof and/or sodium mixtures.

30. A pharmaceutical composition according to claim 19, characterized in that, tonicity adjusting agent can be selected from sodium chloride, mannitol, dextrose, sodium gluconate, sodium pyruvate an/or potassium chloride, glucose, lactose, sucrose, trehalose, xylitol, sorbitol and/or isomaltol.

31. A pharmaceutical composition according to any one of claims 1 to 5, characterized by comprising a sterile isotonic saline solution to achieve desired tonicity of the dosage form.

32. A pharmaceutical composition according to claim 19, characterized in that, surfactants can be ionic or non-ionic surfactant which is safe for oral or nasal inhalation.

33. A pharmaceutical composition according to claim 32, characterized in that, surfactant can be selected from tyloxapol, polysorbates, polysorbate 20, polysorbate 60, polysorbate 80, lecithin, vitamin E TPGS, macrogol hydroxystearates and/or macrogol-15-hydroxystearate.

34. A pharmaceutical composition according to claim 19, characterized in that, taste masking agent can be selected from group of pharmaceutically acceptable sweeteners comprising saccharin, aspartame, cyclamate, sucralose, acesulfame, neotame, thaumatin, neohesperidine and/or salts or solvates thereof.

35. A pharmaceutical composition according to claim 19, characterized in that, taste masking agent can be sodium salt of saccharin or potassium salt of acesulfame.

36. A pharmaceutical composition according to claim 19, characterized in that, taste masking agent can be sucrose, trehalose, fructose, lactose, xylitol, mannitol and/or isomalt.

37. A pharmaceutical composition according to claim 19, characterized in that, taste masking agent can be selected from pharmaceutically acceptable surfactants, alkaline earth metal salts, organic acids and/or amino acids.

38. A pharmaceutical composition according to claim 37, characterized in that, taste masking agent can be citric acid, lactic acid and/or arginine.

39. A pharmaceutical composition according to claim 19, characterized in that, aromatic flavour can be selected from essential oils.

40. A pharmaceutical composition according to claim 39, characterized in that, aromatic flavour can be menthol, thymol or cineol.

41. A pharmaceutical composition according to claim 19, characterized in that, wetting or dispensing agents can be selected from poloxamers, oleic acid or its salts, lecithin, hydrogenated lecithin, sorbitan fatty acid esters, oleyl alcohol, phospholipids including but not limited to phosphatidylglycerol, phosphatidylcholine, polyoxyethylene fatty alcohol ethers, polyoxypropylene fatty alcohol ether, polyoxyethylene fatty acid ester, glycerol fatty acid esters, glycolipid such as sphingolipid and sphingomyelin, polyoxyethylene glycol fatty acid ester, polyol fatty acid esters, polyethylene glycol glycerol fatty acid esters, polypropylene glycol fatty acid esters, ethoxylated lanolin derivatives, polyoxyethylene fatty alcohol, polyoxyethylene sorbitan fatty acid esters, polyoxyethylene stearate, propylene glycol alginate, dilauryldimethylammonium chloride, D-a-tocopheryl-PEG 1000 succinate, Polyoxy 40 stearate, polyoxyethylene-polyoxypropylene block copolymers, polyoxyethylene vegetable oils, fatty acid derivatives of amino acids, glyceride derivatives of amino acids, benzalkonium chloride and/or bile acids.

42. A pharmaceutical composition according to any one of the preceding claims for use in the treatment of viral lung diseases including COVID-19 disease caused by Severe Acute Respiratory Syndrome-Coronavirus-2 (SARS-CoV-2).

43. A pharmaceutical composition according to any one of the preceding claims, characterized in that, it is single use, or multi-use dosage.

44. A pharmaceutical composition according to any one of claims 1 to 5, characterized in that, mass median aerodynamic diameter (MMAD) value is between the range of 1-7 μm.

45. A pharmaceutical composition according to any one of claims 1 to 5, characterized in that mass median aerodynamic diameter (MMAD) value is 5.3 μm

46. A pharmaceutical composition according to any one of claims 1 to 5, characterized in that, mean fine particle fraction (FPF) value is between the range of 10-60%.

47. A pharmaceutical composition according to any one of claims 1 to 5, characterized in that, mean fine particle fraction (FPF) value is 44%.

48. A pharmaceutical composition comprising favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, or pharmaceutically acceptable derivatives thereof for use in the treatment of viral lung diseases including COVID-19 disease caused by Severe Acute Respiratory Syndrome-Coronavirus-2 (SARS-CoV-2) characterized in that, it is locally administered to the lung by means of soft mist inhaler, or vibrating mesh technology nebulizer.

49. A pharmaceutical composition comprising favipiravir, mannitol, hydroxychloroquine, umifenovir, molnupiravir, pimodivir, and/or remdesivir, and/or their water-soluble salt forms, and/or their water-soluble cyclodextrin complexes, and/or their water-soluble forms, or pharmaceutically acceptable derivatives thereof, which are dissolved in a carrier solution, in order to locally administer it to the lungs for the treatment of viral lung diseases including COVID-19 disease caused by Severe Acute Respiratory Syndrome-Coronavirus-2 (SARS-CoV-2), acute lung diseases, and/or chronic lung diseases by means of soft mist inhaler or active vibrating mesh technology nebulizer or passive vibrating mesh technology nebulizer through inhalation.
